(19) United States
(12) Patent Application Publication
(10) Pub. No.: US 2021/0183473 A1
(43) Pub. Date: Jun. 17, 2021

(54) SYSTEM AND METHODS FOR GRAPHIC ENCODING OF MACROMOLECULES FOR EFFICIENT HIGH-THROUGHPUT ANALYSIS

(71) Applicant: STC.UNM., Albuquerque, NM (US)

(72) Inventors: Trilce PROCYON ESTRADA-PIEDRA, Albuquerque, NM (US); Jeremy BENSON, Albuquerque, NM (US); Hector CARRILLO-CABADA, Albuquerque, NM (US); Ewa DEELMAN, Albuquerque, NM (US); Michela TAUFER, Newark, DE (US)

(21) Appl. No.: 16/761,663

(22) PCT Filed: Nov. 8, 2018
(86) PCT No.: PCT/US2018/059901

§ 371 Date: May 5, 2020

Related U.S. Application Data
(60) Provisional application No. 62/583,391, filed on Nov. 8, 2017.

(51) Int. Cl. G16B 45/00; G16B 15/30; G16B 15/20; G16B 40/00

(52) U.S. Cl. CPC G16B 45/00; G16B 40/00; G16B 15/20; G16B 15/30

(57) ABSTRACT

The present invention is directed to a system and methods of predicting protein function through a process of encoding protein structural information into a computer readable format and the use of a convolutional neural network designed to recognize such encoded format.

### CROSS-REFERENCE TO RELATED APPLICATIONS

This invention claims priority to U.S. Provisional Patent Application Ser. No. 62/583,391 filed Nov. 8, 2017, incorporated herein by reference in its entirety.

### STATEMENT REGARDING FEDERALLY SPONSORED RESEARCH

This invention was made with government support under Grant No's. 1453430 and 1741057 awarded by The National Science Foundation. The government has certain rights in the invention.

### FIELD OF THE INVENTION

The invention generally relates to the analysis of protein folding and structure. More specifically the invention is related to a system and methods of encoding protein secondary structure and tertiary structure information into a computer readable format for use with a neural network for high-throughput analysis.

### BACKGROUND OF THE INVENTION

Proteins are biopolymers that may comprise from a few dozens to several thousand amino acids connected together through peptide bonds in a linear sequence referred to as the primary structure of a protein. Shorter domains of regularly folded sequences (e.g., α-helices, β-sheets, and reverse turns) form the secondary structure of a protein. Further folding of the secondary structure shapes the protein into a unique three-dimensional structure—known as the tertiary structure—in order to interact with their environment (e.g., interaction with other proteins, perform a structural or enzymatic function). Accordingly, identifying and understanding the secondary and tertiary structures are fundamental to identifying the function of a given protein.

Commonly used techniques of predicting protein function includes various homology-based methods based on the idea that proteins or polynucleotides with similar sequences (i.e., homologous sequences) share similar functions. For example, DNA, RNA, or proteins may be represented by their amino acid residues or nucleotide sequences, that is, a succession of letters GACT for DNA, GACU for RNA, and the one-letter codes for the 20 natural amino acids for proteins. These aligned sequences may be represented through matrices in which each sequence corresponds to a row and multiple rows of sequences may be aligned in columns. Pairwise sequence alignment may be then performed using dynamic programming (e.g., Smith-Waterman algorithm, Needleman-Wunsch algorithm, both with a time complexity of O(nm), where n and m are sequence length for a pair sequence alignment). Ultimately, such homology-based methods may identify shared structural motifs that may be indicative of a certain shared protein function. However, this is not always true. Consequently, the alignment of the sequences alone may not be enough to accurately predict the function of a protein. Instead, accurate prediction of protein function may require also the knowledge of the folding patterns of the three-dimensional structure of the protein.

Structural comparison of proteins is a critical aspect of multiple research problems, including protein annotation and protein structure prediction. Structure-based function prediction often outperforms sequence-based methods because structural homologues may include similar folding patterns even though the similarity of the amino acid sequence may be completely undetectable.

Generally, structural comparison of proteins combines primary amino acid sequence information with the secondary and tertiary structure of the protein or polynucleotide molecule, and it is considered as the standard practice for homology-based structure and function prediction. But thoroughly comparing protein structures that may range in size from tens to several thousand amino acids may be computationally expensive. Moreover, for high-throughput analysis and identification of homologous structures, the alignment and comparison must be performed for multiple macromolecules at a time. Further, the alignment and structural comparison must be carried out in a pair-wise manner, which limits the opportunity for executing the analysis of structures concurrently, that is, the analysis of a specific protein depends on its comparison to a large database of other protein structures. This all-to-all structural alignment is a major bottleneck in the scalability of homology-based approaches. Furthermore, as the number of proteins increases over time (e.g., with the advancing of crystallography and NMR techniques), more scalable analysis techniques are needed to take advantage fully of the information embedded in new and existing proteins.

Previous attempts to overcome the scalability issue include the use of machine learning (“ML”) methods such as convolutional neural networks (“CNNs”) and deep learning (“DL”). In particular, these ML methods may be the de-facto techniques in computer vision and image processing and may solve previously open problems—such as object recognition—with high accuracy. And as the use of ML methods increase, DL methods have been increasingly applied to scientific applications, including structural biology. However, as the function of a protein directly depends on the three-dimensional structure, computational approaches for protein function prediction—and, more generally, protein analysis—based on ML may be limited by the method in which proteins are represented. Inherent differences between proteins, such as length, location of structural motifs, and different folding conformations, present significant challenges for representing proteins in a manner that may be adequately analyzed by machine learning methods.

Accordingly, there is a need for a scalable method of encoding structural information for a given protein into an easily searchable computer readable format that identifies and presents protein structural information without the need for pairwise structural alignments and/or complicated homology calculations. The present invention satisfies this need.

### SUMMARY OF THE INVENTION

The present invention is directed to a system and methods of graphically encoding the secondary and tertiary structural information of a protein into a computer readable representation (e.g., an image or tensor) that may significantly increase the high-throughput techniques for predicting protein function.

Advantageously, embodiments of the invention may be invariant to the protein size (i.e., number of amino acid residues forming the protein). That is to say, even though proteins may vary in size from tens to several thousand residues, embodiments of the invention may graphical encode structural representations of a protein regardless of size using a standardized squared distance matrix.

Advantageously, certain preferred embodiments of the invention may display the structural domains and folding motifs of a protein as a pattern in a representation (e.g., an image or tensor) that may be easily searchable by computer neural networks. For example, the computer readable format may include a three-dimensional tensor (i.e., a matrix with one or more dimensions) in which each dimension encodes a color channel in a Red-Green-Blue (RGB) color model.

One certain preferred embodiment of a system and method for graphically encoding secondary and tertiary structural information of a protein into a computer readable representation may include the steps of extracting secondary structural information from a target protein using a Ramachadran Plot, expressing the secondary and tertiary structural information of the target protein using a distance matrix, encoding the secondary and tertiary structural information of the target protein into multiple codified channels to form an image or tensor, formatting the image or tensor into a fixed-size final representation encoding the structural information of the target protein, and analyzing the final representation using machine learning in order to predict or classify protein function.

Certain preferred embodiments of the invention may assign an amino acid of a protein a secondary structure such as an α-helix, a β-strand, a Polyproline PII-helix, a γ′-turn, a γ-turn, a cis-peptide bonds, or indeterminate based on the constraints of the torsion angle phi, φ, (angle between the C-N-CA-C atoms in an amino acid) versus the torsion angle psi, ψ, (angle between the N-CA-C-N atoms in an amino acid) in the Ramachadran plot. Preferably, each of these secondary structures may be represented by a certain color. In one certain preferred embodiment of the invention, the saturation level of each color representing a certain secondary structure may be expressed as [1,sd(i,j),sd(i,j)]Åj ∈ D, [sd(i, j), sd(i, j),1], [1, sd(i,j), 1], [1,1, sd(i, j)], [sd(i, j), 1, 1], [sd(i, j),1, sd(i, j)], and [0,0,0], for red, blue, magenta, yellow, cyan, green, and black, respectively.

Certain preferred embodiments of the invention also may resize the protein representations—that is, the graphically encoded image or tensor—using, for example, bicubic interpolation to produce a dimensionally consistent fixed-sized encoded representation of the secondary and tertiary protein structural information.

Certain embodiments of the invention include also a neural network architecture specifically designed to analyze the encoded structural information of the protein. Advantageously, such embodiments of the neural network architecture handle each color channel independently prior to grouping the individual color channel into a single image or tensor.

These and other exemplary features and advantages of the present invention will become clear from the following description with reference to the accompanying drawings.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the presented detailed description. The description and accompanying drawings may highlight a certain specific example, or a certain aspect of the invention. However, one skilled in the art will understand that portions of the example or aspect may be used in combination with other examples or aspects of the invention.

FIG. 1 illustrates one certain preferred embodiment of a method of the invention;

FIG. 2 illustrates one encoding procedure for the gene V protein (PDBid: 1AE2) according to one embodiment of the present invention;

FIG. 3A illustrates a 3-D cartesian representation of human alpha-lactalbumin protein (PDBid: 1A4V);

FIG. 3B illustrates a Multi-fold representation of human alpha-lactalbumin protein (PDBid: 1A4V);

FIG. 3C illustrates a Surface representation of human alpha-lactalbumin protein (PDBid: 1A4V);

FIG. 3D illustrates a representation of human alpha-lactalbumin protein (PDBid: 1A4V) using an embodiment of the present invention;

FIG. 4A illustrates a Multi-fold representation of protein (PDBid: 1A5N);

FIG. 4B illustrates an encoded representation of protein (PDBid: 1A5N) using one embodiment of the present invention;

FIG. 4C illustrates a Multi-fold representation protein (PDBid: 2A9H);

FIG. 4D illustrates an encoded representation of protein (PDBid: 2A9H) using an embodiment of the present invention;

FIG. 4E illustrates a Multi-fold representation of protein (PDBid: 1AGF);

FIG. 4F illustrates an encoded representation of protein (PDBid: 1AGF) using an embodiment of the present invention;

FIG. 4G illustrates a Multi-fold representation of protein (PDBid: 1A67);

FIG. 4H illustrates an encoded representation of protein (PDBid: 1A67) using an embodiment of the present invention;

FIG. 4I illustrates a Multi-fold representation of protein (PDBid: 1A8A);

FIG. 4J illustrates an encoded representation of protein (PDBid: 1A8A) using an embodiment of the present invention;

FIG. 4K illustrates a Multi-fold representation of protein (PDBid: 3A9Q);

FIG. 4L illustrates an encoded representation of protein (PDBid: 3A9Q) using an embodiment of the present invention;

FIG. 4M illustrates a Multi-fold representation of protein (PDBid: 4A8Z);

FIG. 4N illustrates an encoded representation of protein (PDBid: 4A8Z) using an embodiment of the present invention;

FIG. 4O illustrates a Multi-fold representation of protein (PDBid: 3AE1);

FIG. 4P illustrates an encoded representation of protein (PDBid: 3AE1) using an embodiment of the present invention;

FIG. 5 illustrates a dataset breakdown of proteins of the PDB according to protein classification;

FIG. 6 illustrates one embodiment of the invention using a slit-input graphic encoding of macromolecules used for classification/prediction of protein function;

FIG. 7 illustrates a comparison a single channel encoding method and a three-channel encoding method according to the present invention;

FIG. 8 illustrates a confusion matrix for protein classification using one embodiment of the invention;

FIG. 9A illustrates a screenshot from a video of compiled images of an encoded protein showing movement of the protein over time as changes in color and color intensities (see closed arrows);

FIG. 9B illustrates a screenshot of a video of a protein trajectory (the movement of a protein over time) of the same protein in FIG. 9B at the same time point (see open arrow);

FIG. 9C illustrates a screenshot from a video of compiled images of an encoded protein showing movement of the protein over time as changes in color and color intensities (see closed arrows);

FIG. 9D illustrates a screenshot of a video of a protein trajectory (the movement of a protein over time) of the same protein in FIG. 9C at the same time point (see open arrow);

FIG. 9E illustrates a screenshot from a video of compiled images of an encoded protein showing movement of the protein over time as changes in color and color intensities (see closed arrows);

FIG. 9F illustrates a screenshot of a video of a protein trajectory (the movement of a protein over time) of the same protein in FIG. 9E at the same time point (see open arrow);

FIG. 9G illustrates a screenshot from a video of compiled images of an encoded protein showing movement of the protein over time as changes in color and color intensities (see closed arrows);

FIG. 9H illustrates a screenshot of a video of a protein trajectory (the movement of a protein over time) of the same protein in FIG. 9G at the same time point (see open arrow); and

FIG. 10 illustrates an exemplary computer system for use with embodiments of the invention.

### DETAILED DESCRIPTION OF THE INVENTION

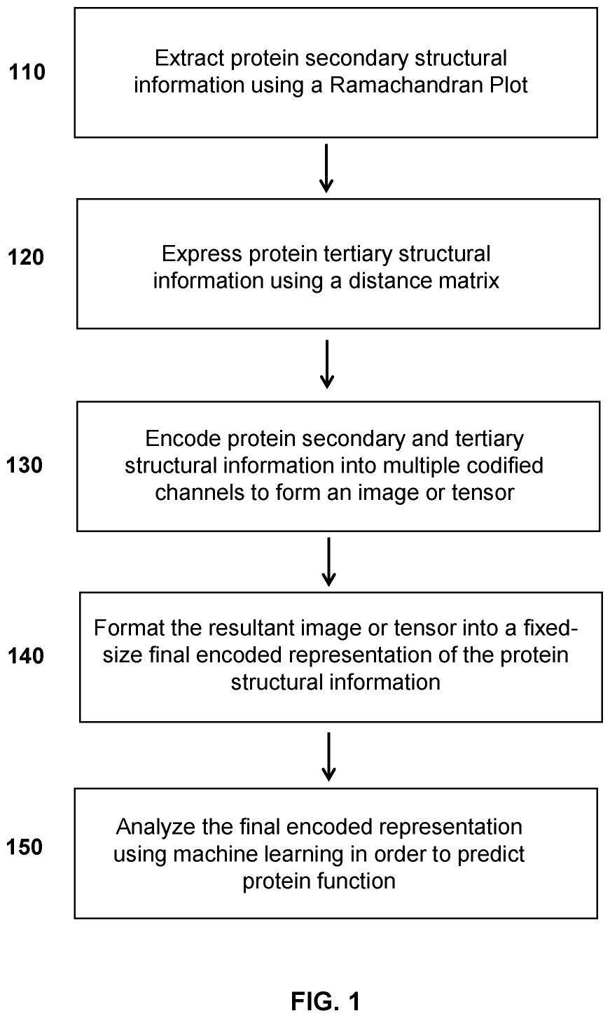

The present invention is directed to a system and methods of graphically encoding the secondary and tertiary structural information of a protein into a computer readable representation. FIG. 1 generally illustrates one such embodiment that may include the steps of extracting secondary structural information of a protein 110, expressing tertiary structural information of the same protein using a distance matrix 120, encoding both the secondary and tertiary structural information of the protein in multiple codified channels to form an image or tensor 130, formatting the resultant image or tensor into a fixed-size encoded representation of the protein structural information 140, and analyzing the fixed-size encoded representation using machine learning techniques to predict protein function 150.

Certain preferred embodiments of the invention may identify and extract the basic molecular structures (e.g., secondary structure) forming the protein 110. Preferred embodiments of the invention may identify and extract such structural information through the analysis of an x-ray crystal structure or, more preferably, the backbone dihedral angles (that is, angles between two intersecting planes that have two atoms in common) of the amino acid residues in the macromolecular structure. The Ramachandran plot, originally developed by G. N. Ramachandran, C. Ramakrishnan, and V. Sasisekharan in 1963, determines the energetically allowable regions for the torsion angle phi, φ, (angle between the C-N-CA-C atoms) versus the torsion angle psi, ψ, (angle between the N-CA-C-N atoms), and omega Ω (usually restricted to be 180 degrees for the typical trans case or 0 degrees for the rare cis case) for each amino acid of a protein sequence. Each amino acid in the protein then may be associated with one of six types of secondary structures: α-helix, β-strand, Polyproline PII-helix, γ′-turn, γ-turn, and cis-peptide bonds based on the constraints of the torsion angles (φ, ψ, and Ω) as described by the Ramachandran plot.

According to step 120, certain embodiments of the invention next may establish a spatial correlation between the different secondary structures in the protein in order to provide, for example, tertiary structural information. This may include the use of a distance matrix such that for a protein with M Alpha Carbon atoms Cα, the distance matrix may be defined as a squared matrix D of size M×M, where the element D(i, j) corresponds to the distance between Alpha Carbon atoms Cαᵢ and Cαⱼ, thus making the distance matrix symmetric. Further, the distance matrix may not be restricted to a particular distance metric; rather, any metric or correlation coefficient may be used for this purpose (e.g., Euclidean, squared Euclidean, Minkowsky, Chevychev, cosine, spearman, or hamming). Certain preferred embodiments of the invention may use the Euclidean distance between alpha carbon atoms in the backbone to provide information regarding both the secondary and tertiary structure of the protein.

Next, as shown in step 130, the extracted secondary structures and distance matrix information may be encoded into a representation of the protein such as an image or a tensor. Certain preferred embodiments of the invention may use a tensor dimension of M×M×3, where M is the number of amino acid residues in the protein, and 3 indicates the Red-Green-Blue channels (although more or less color channels may be used with the invention) in the image. Using this technique, a color code may be used to identify and display secondary structures, intensity, or color saturation to proportionally represent distances between atoms in the protein. For example, in step 110, amino acid residues may be classified according to their dihedral angles into one of six secondary structures. An RGB color model including six arbitrary colors may be then used to differentiate each secondary structure as a certain color. For example, α-helix, red; β-strand, blue; Polyproline magenta; γ′-turn, yellow; γ-turn, cyan; and cis-peptide bonds, green. A seventh color (e.g., black) may be assigned to amino acids that do not fall within one of the six secondary structures or may represent additional protein structure information as discussed below. Consequently, the structural information of a protein may be encoded into an image representation by defining function sd(i,j) , where sd(i,j) may defined as a normalized distance function that returns a value between 0 and 1 proportional to the distance between Alpha Carbon atoms i and j in the protein.

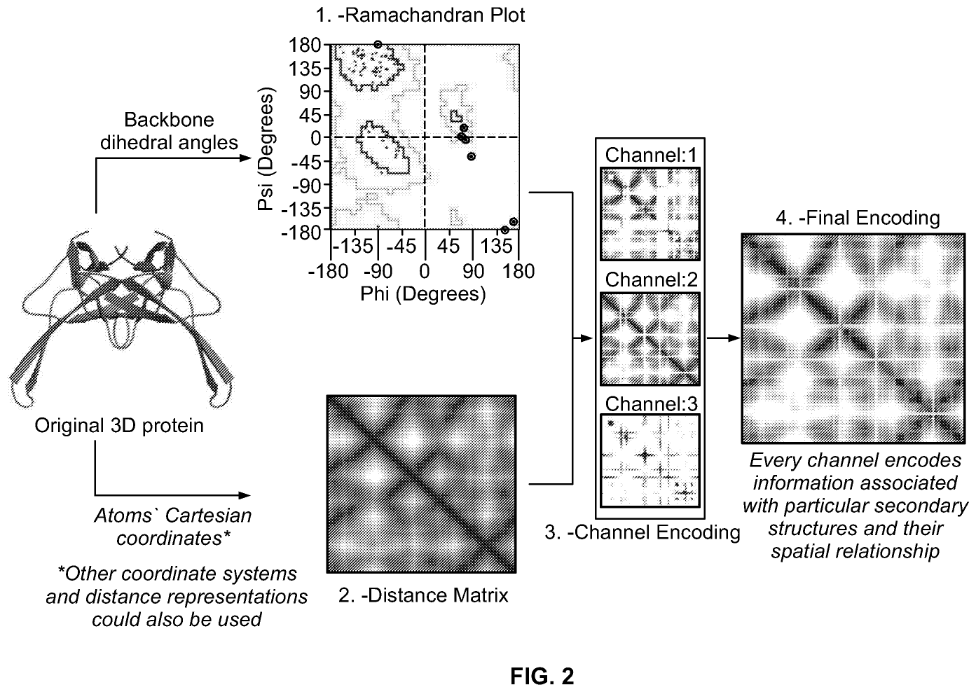

As is further illustrated in FIG. 2, certain preferred embodiments of the invention may use a three color-channel image in order to identify a particular amino acid residue as one of seven possible conformational structures (six secondary structures and an unidentified structure) and a color saturation level of each channel associated with a particular type of secondary structure may be assigned to the amino acid residue. For example, for a red α-helix in position i, the saturation for channels red, green, and blue may be [1, sd(i,j), sd(i,j)] Åj ∈ D where sd(i,j) is the distance function between the i^{th} and j^{th} residues in the distance matrix D. In the same way, the saturation levels for each of the remaining six structures may be depicted in, for example, blue, magenta, yellow, cyan, green, and black are [sd(i, j), sd(i, j),1], [1, sd(i, j),1], [1,1, sd(i, j)], [sd(i, j), 1, 1], [sd(i, j),1, sd(i, j)], and [0,0,0], respectively. This process may produce symmetric representation (e.g., an image or tensor) that may visually highlight the secondary and tertiary structure of a protein so that small differences between similar structures may be noticeable by sharp changes of color, such as, for example, when a helix unfolds into a γ-turn and changes from red to cyan. It will be appreciated that the number of color channels that may be used in a given image or tensor may be adjusted by the user to accommodate information of any relevant biophysical parameter of the protein. That is to say, any property of the protein structure that may be extracted from a residue of the protein may be included in a tensor as another color channel. For example, in addition to the secondary and tertiary structural information, an image may encode also the electromagnetic charge, residual energy, or the hydrophobicity of a protein using various color channels. Such additional protein structural information may be extracted from, for example, crystalized protein structures or through the use of computer simulations (e.g., Molecular Dynamic simulations) that directly incorporate specific force fields and electrodynamics during the simulation time.

After formation of the representation of the protein from the secondary and tertiary structural information, the resultant image or tensor may be resized (e.g., through the application of, for example, a bicubic interpolation, bilinear interpolation, or nearest neighbors interpolation. In preferred embodiments of the invention, the image or tensor may be resized through the use of bicubic interpolation (i.e., extension of cubic interpolation for interpolating data points on a two-dimensional regular grid, this process results in smooth transitions between the original grid—in this case an image, and its expanded or reduced version) to produce a dimensionally consistent image or tensor output regardless of the length of the protein such as is described in Gao et al., Bilinear and bicubic interpolation methods for division of focal plane polarimeters. Opt Express. 2011 Dec. 19; 19 (27):26161-73. doi: 10.1364/OE.19.026161. For example, assuming a new image size N, the output is a N×N×Z tensor, where N represents the new size of the image that may be smaller or larger than the original image N, and Z is the number of color channels used in the encoding the structural information into an image. The number of color channels used to encode the structural image may vary according to user preference (e.g., 1, 2, 3, 4, 5, 6, 7, 8, 9, 10, etc. color channels). In preferred embodiments of the inventions, at least three color channels—red, green, and blue—may be used to encode the structural information into an image. The output—either an image or tensor—may encode one or more amino acid residue per pixel, or alternatively, may use multiple pixels to encode one amino acid residue so long as the same ratio is used for each output image or tensor.

Further, the size of N may be chosen by the user to optimize different performance metrics. For example, N may be equal to the number of amino acid residues in the longest protein in a dataset to optimize the fidelity of encoding the structural information. In certain embodiments of the invention, N may equal the average number of amino acid residues in the dataset that may result in a tradeoff between fidelity and efficiency of encoding the structural information. In further embodiments of the invention, N may be set to an arbitrary small size in order to maximize efficiency of encoding the structural information. In certain preferred embodiments of the invention, N was set to 227, which is a standard size for image processing.

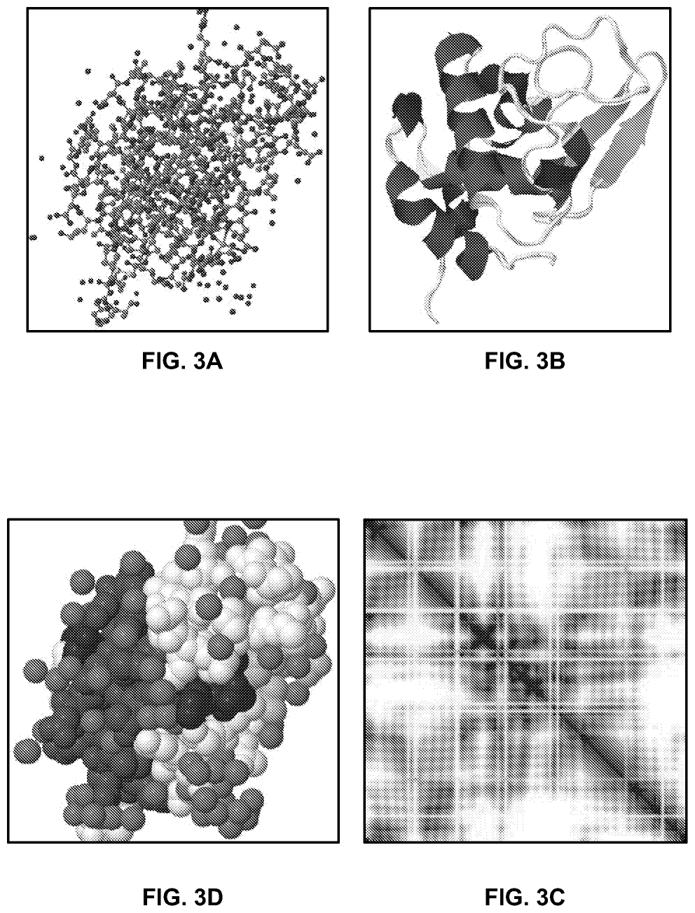

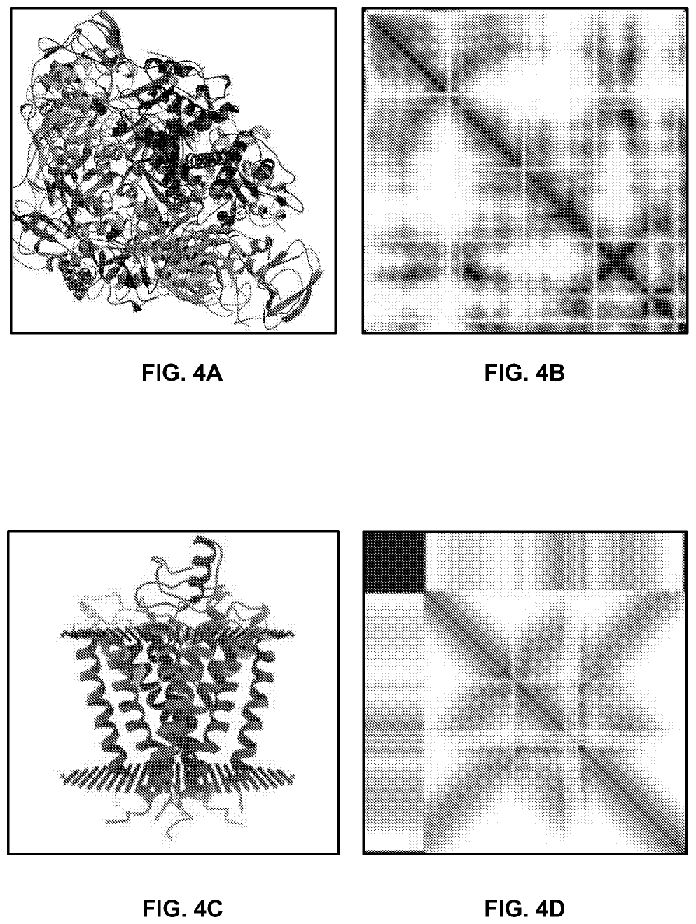

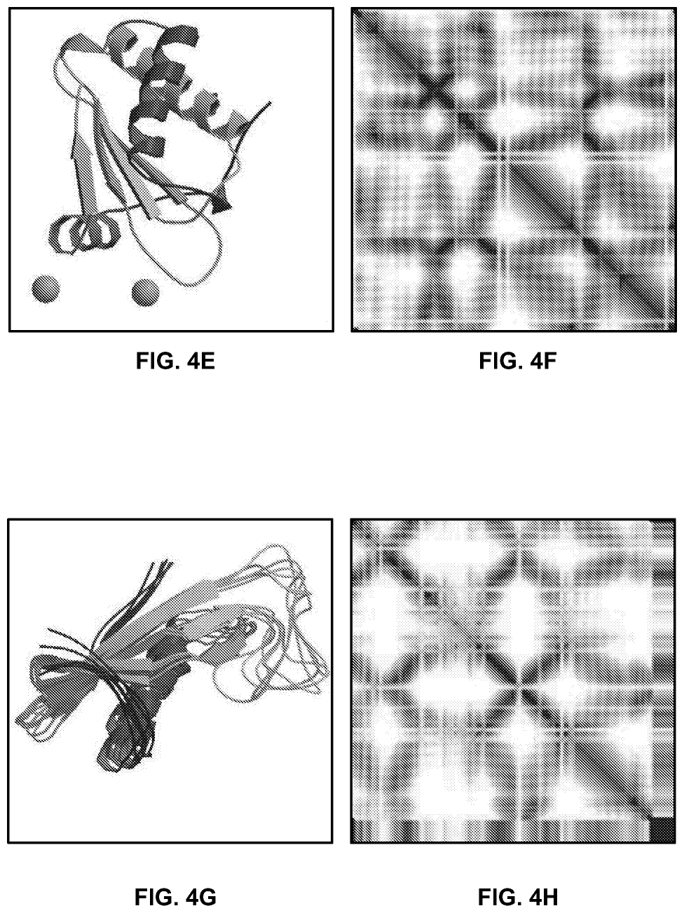

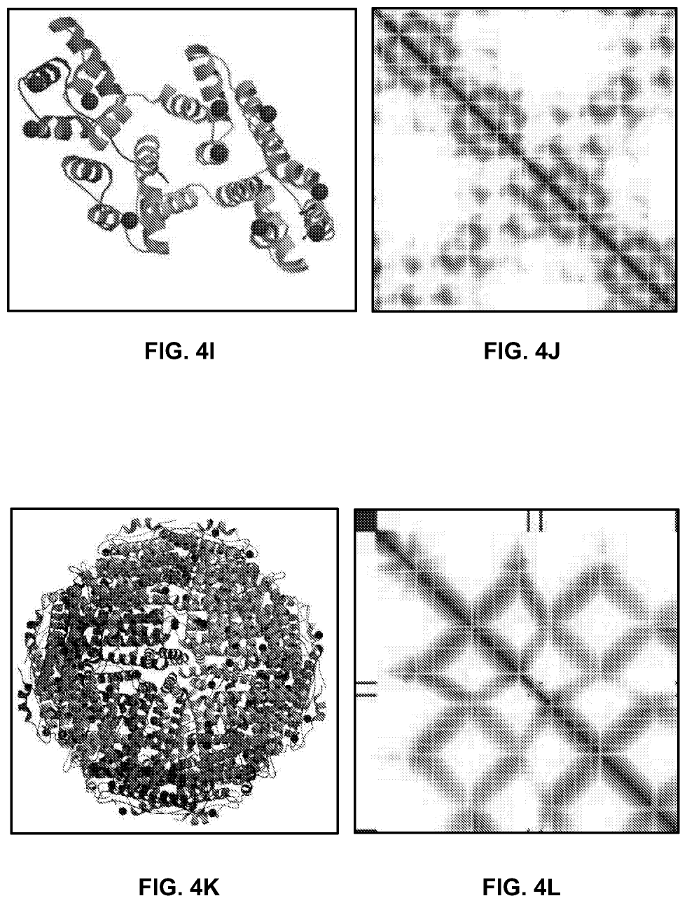

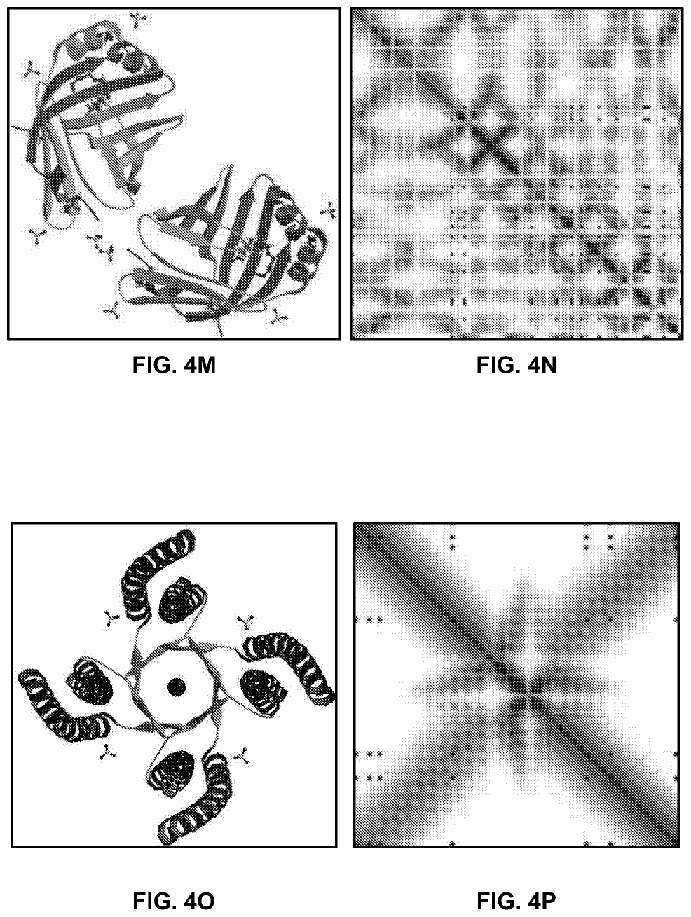

FIG. 3A-3D and FIG. 4A-4O illustrates examples of various macromolecules shown in a three-dimensional representation (a multi-fold representation) compared to a representation created using the inventive methods described in this Application. A comparison of these images clearly demonstrates the ability of the encoding methods of the invention to differentiate certain patterns at various levels of granularity in each image. In this way, the inventive encoding methods transform traditional structural biology problems into an image pattern recognition and may facilitate the use of sophisticated image processing and machine learning techniques for analysis and prediction of protein function.

Embodiments of the graphical encoding methods used to identify structural information necessary to perform basic protein function prediction were analyzed using a dataset consisting of 62,991 proteins from the Protein Data Bank. The protein data bank format (“PDB”) provides a standard representation for macromolecular structural data derived from X-ray diffraction and NMR studies. A PDB file encodes a protein as a sequence of atoms, their type, and their three-dimensional coordinates. These representations may be converted easily into a representation as described in this Application. The structural encoding proteins in the test dataset range in size from less than 100 non-hydrogen atoms to more than 50,000 atoms and the mean size being 6508 atoms with a standard deviation of 19495. The mean resolution of the proteins in the PDB is about 2.2 Angstroms, with a 1.7 standard deviation. The main source organism in this dataset includes Homo Sapiens, Escherichia coli, Mus musculus, Saccharomyces cerevisiae, Rattus norvegicus, and Mycobacterium tuberculosis among others. FIG. 4A-4O illustrates multiple examples of proteins in the test dataset converted into a representation using the graphic encoding method.

The protein function prediction capabilities of the invention were tested by first obtaining Gene Ontology (“GO”) terms through the RCSB Protein Data Bank and their biological details report of each protein in the test database. GO terms are established by the Gene Ontology Consortium (GOC) (Ashburner et al., Gene Ontology: tool for the unification of biology., The Gene Ontology Consortium. Nat. Genet. 25, 1 (2000)) GOC provides GO terms as a standardized and consistent way of describing and documenting gene products across databases. The GO project comprises three structured ontologies with a well-defined vocabulary to express gene product properties over three domains: cellular component, molecular function, and biological process in a species-independent manner. Terms in the cellular component describe the parts of a cell or its extracellular environment, for example a ribosome. Terms in the molecular function describe activities that are performed by individual gene products or assembled complexes. Examples of such activities include binding or catalysis. Finally, terms identifying biological processes encompass series of events carried out by molecular function with a well-defined beginning and end.

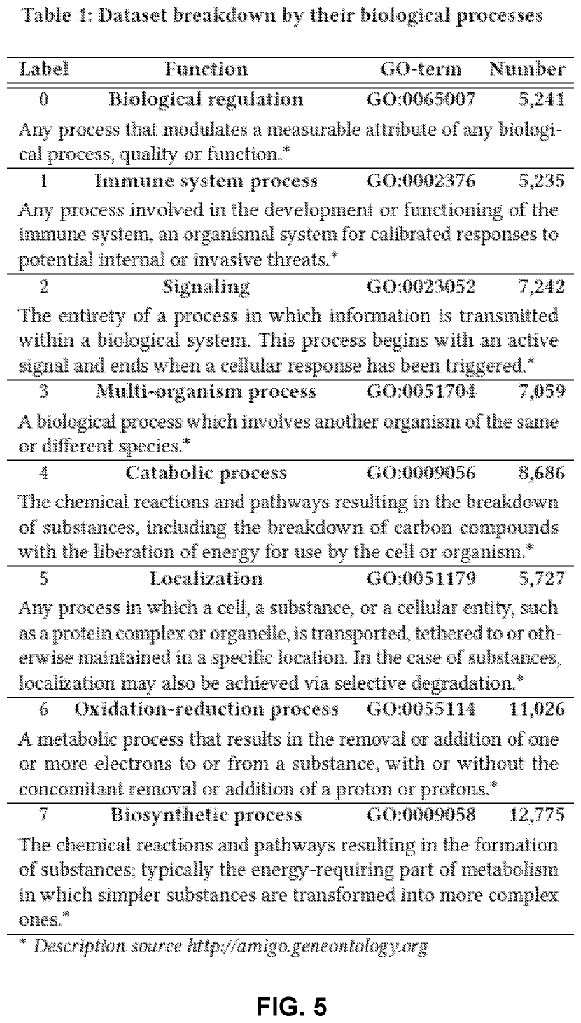

Next, proteins in the test database were assigned a label according to a specific function using a biological process taxonomy provided by RCSB-PDB. Eight biological processes with the greatest number of proteins were selected from this taxonomy (i.e., more than 5,000) and used for protein classification. The protein function classification is illustrated in FIG. 5 and includes the following: Label 0 contains proteins involved in biological regulation, this class is characterized by GO:0065007 and contains 5,241 proteins. Label 1 is characterized by GO:0002376, indicative of immune system processes with 5,235 proteins. Label 2 is characterized by GO:0023052 for signaling with 7,242 proteins. Label 3 is characterized by GO:0051704 and represents multi-organism processes with 7,059 proteins. Label 4 contains 8,686 proteins involved in catabolic processes and is characterized by GO:0009056. Label 5 is characterized by GO:0051179 for localization and contains 5,727 proteins. Label 6 is characterized by GO:0055114 indicative of oxidation-reduction processes with 11,026 proteins. Label 7 contains 12,775 proteins involved in biosynthetic processes characterized by GO:0009058.

The graphically encoded representations were then examined using two known machine learning techniques, and in particular, a convolutional neural network, to predict protein function. A convolutional neural network, also known as a CNN, is a mathematical construction that trains complex non-linear functions out of linear compositions. CNNs handle matrix-oriented input, usually ingesting images, and produce a classification output. Convolutions are employed to preserve spatial relationships between pixels and learn important image features, such as edges, flattened areas, or other patterned shapes. A CNN is usually composed of a variety of convolutions (i.e., a filter kernel is convolved with an input), pooling (i.e., some input is down-sampled via some maximum or averaging over a neighborhood of pixels), and dense layers (i.e., a fully connected perception). Activation functions like sigmoid or rectified linear units help to remove noise or smooth the data between layers. Representing secondary and tertiary structural information of proteins as 3D tensors attempts to utilize a CNN's superior capability in identifying spatial relationships, which in this context, translates to identifying structural patterns.

One known CNN used to test the graphical encoding methods is VGG-net. This CNN has been shown previously to be successful for a variety of image classification tasks. In the testing of the graphical encoding methods, the VGG-net was configured to include 8 convolution and 3 fully connected layers, with very small receptive fields of 3×3 in layers that increase in width by a factor of two, starting from 64. The CNN includes also max-pooling layers (i.e., layers that reduce the dimensionality of the convolution by using a summarization function, for example a Maximum value) after each convolution layer.

Another CNN—Google's Inception-v3 network—is a general-purpose image recognition system trained for the ImageNet large visual recognition challenge to discriminate entire images into 1,000 classes. The architecture of the inception network is a series of inception modules, which are simply sets of convolutional filters concatenated together in order to capture information at varying kernel sizes (which is to say that, at each layer, the input is convolved with multiple kernels that vary in width and height; ultimately, the results of these convolutions are grouped together and sent to the next layer). Building such a deep network that may be practical for predicting protein function may require a very large number of labeled images (i.e., the original Inception network for ImageNet was trained on 1.2 million images, with 50,000 images for validation and 100,000 images for testing). Consequently, the length of time of the training phase may be highly dependent on the compute capabilities of the machine. Yet, once trained, the networks may be used to identify salient features from new classes and may be updated for a different classification task. This transfer learning—that is, the process of taking an existing neural network that has been trained on some dataset and re-purposing it for a new classification task. Specifically, the final layer of these networks may be updated to handle new classes—builds upon previous learned knowledge for a new task without the need to completely retrain the network. Another pre-trained network available for retraining is MobileNet (Howard et al., MobileNets: Efficient Convolutional Neural Networks for Mobile Vision Applications. (04/2017), a streamlined deep architecture designed for mobile and embedded systems.

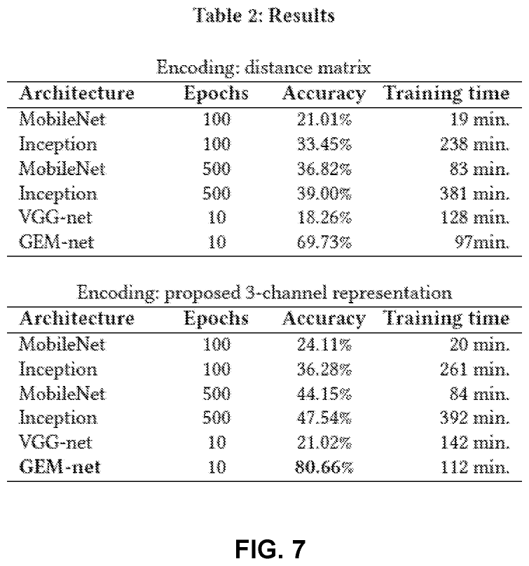

Initial testing of the graphical encoding of the proteins in the PDB leveraged transfer learning by using both Inception-v3 and MobileNet. Although the images used to train these networks are significantly different from the data sets created using the inventive methods according to the invention, the resulting classifiers may group images with reasonable accuracy (FIG. 7). These results indicate that the graphical encoding methods highlight the diverse features of the dataset that permits these pre-trained networks to quickly and effectively sort through the data.

The above testing indicates the encoding methods are useful for input into general purpose and pre-trained neural networks in predicting protein function. Yet, a CNN configured with an architecture to analyze specifically the graphically encoded representations may improve upon predicting protein function. For example, VGG-net and other networks used in transfer learning all intake a 3-color channel image and apply convolutions and other operations directly to the input color channels. This immediate convolution means that the input color channels are both handled and combined together for further downstream processing. However, according to the inventive encoding methods presented in this Application, the input channels are not immediately combined; rather, each of the color channels is separately maintained and represents a different secondary structure. Each color channel may be then treated independently of one another using learning filters that may be relevant for each type of secondary structure. Additionally, embodiments of the invention may make use of resonant units such that the input may be propagated beyond the first convolution to a subsequent layer, rather than perturbing the input and losing it as is the case in other known CNNs.

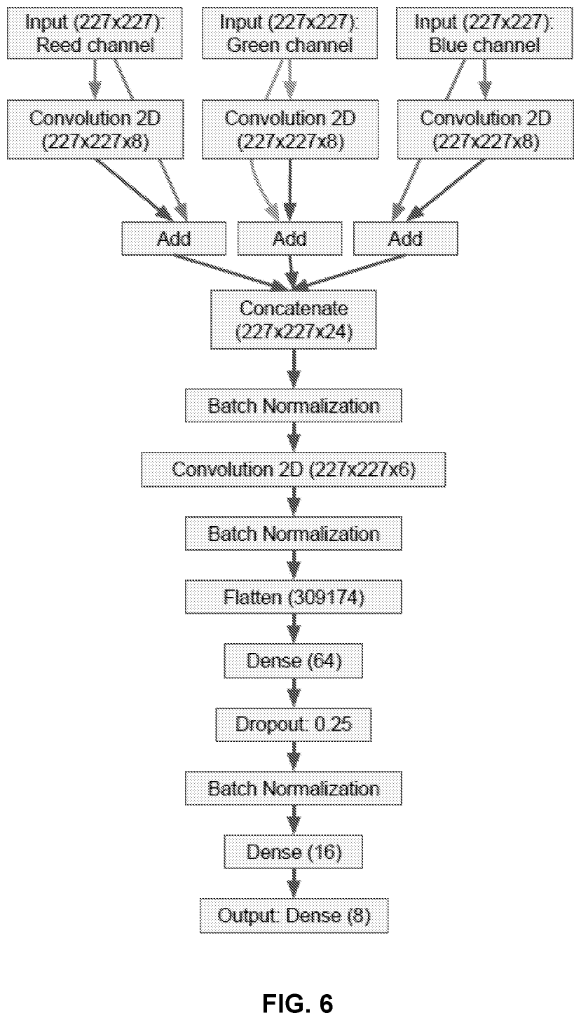

One certain preferred embodiment of an inventive CNN—termed Graphic Encoding of Macromolecules Network, or GEM-net—includes a split-input resonant architecture designed to extract the most information from each channel, independently, and group the information thereafter. FIG. 2 and FIG. 6 depicts the general architecture of GEM-net that initially treats each color channel separately (e.g., each channel is filtered through a separate convolution 2D layer) and then the channels are combined to form a tensor. The tensor may be further processed through a series of convolutional and fully connected layers. Batch normalization between layers serves to denoise the intermediate output tensors and help with both convergence time and final classification accuracy.

GEM-net and several neural network architectures described above were used to evaluate two different encoding methods: the first encoding method includes only the distance matrix of the protein (i.e., information regarding tertiary structure is encoded into a single channel), and the proposed encoding mechanism that may encode the secondary and tertiary structural information into one or more color channel images, and more preferably, three or more color-channel images, where each color represents a specific secondary structure and each color saturation represents the tertiary structure of a protein. All tests included a 5-fold cross validation, which splits the dataset into 5 disjoint partitions, each worth about 20% of the data. Training of the networks may then be conducted with 4 out of 5 partitions (i.e., 80%) and testing is done with the unseen partition. The process may be repeated for a total of 5 times, using each time a different set of partitions for training and testing. Through this process, every protein in the dataset may be used for training four times and for testing once. A learning rate of 0.005—which is standard for smaller size datasets—was used, a batch size of 100, and cross-entropy as a loss-function. The number of epochs may vary between architecture.

The pre-trained networks required longer training periods because these networks only change weights in the last layer and use features learned from general image classification in the other layers. The trained networks converged quite quickly (within 10 epochs), and further training steps only increased overfitting. Both data representations are square images of size 227. The hardware used to construct GEM-net include, for example, an Intel Xeon 8 core E5-1620 v4 processor at 3.50 GHz and a GPU Tesla K80. A summary of the results of the training is presented in FIG. 7 and includes performance metrics such as the mean accuracy and training times in minutes.

The final class assignments of protein function were made based on the GO term classification of a protein to validate the results. It is noted that none of this information is provided to the inventive classifier. Like many convolutional architectures, the inventive network relies on the images solely to learn distinguishing characteristics from the groups and perform a final classification of protein function. The results shown in FIG. 7 demonstrate that several of these image classifiers may discriminate among the eight classes of protein functions. Surprisingly, these results demonstrate the benefit of utilizing three channels of information (i.e., according to the proposed encoding methods) as opposed of just a single channel (i.e., the distance matrix).

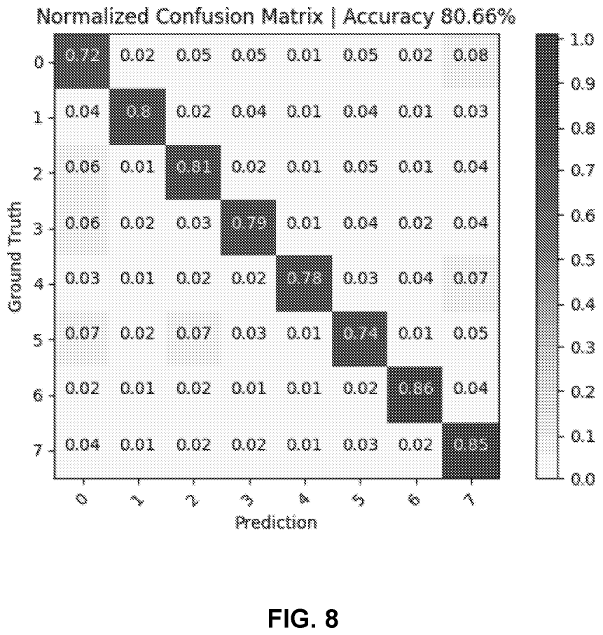

FIG. 8 illustrates a normalized confusion matrix for the prediction of eight protein function classes applied to the PBD test set using the combination of GEM-net and the graphical encoding methods. The confusion matrix is a special instance of a contingency table that describes the performance of the classifier. Every row r represents instances whose correct classification is r. Every column c represents instances that were predicted as being of Class c. Cells in the diagonal indicate correct predictions. Every other cell indicates mistakes in the classification. These results indicate that the proposed graphical encoding methods together with GEM-net and only 10 epochs, produced a prediction accuracy of above 85% for two of the classes (labels 6 and 7) and below 75% for only two of the classes (labels 0 and 5).

Other certain preferred embodiments of the invention may be used in protein folding and/or protein ligand-docking simulations to determine local (i.e. a few amino acid residues close enough in space to have strong energetic interactions) and global (i.e., complete structures that may be acting independently of each other) arrangements of biological or structural significance. Such simulations may include Molecular Dynamics (MD) simulations, which model explicitly (i.e., by modeling each atom as an element with explicit properties such as position, charge, and velocity in the simulation) or implicitly (i.e., by using equations) the movement and interaction of atoms and molecules. MD simulations may incorporate one or more molecules, a force field, and a solvent, in order to simulate inter and intra protein movement and interaction over time. Such folding simulations seek to understand the way in which molecules fold and unfold. Other simulations—docking simulations—seek to understand how and if small molecules—termed ligands—dock into or otherwise interact with target proteins. Understanding the different structures that form during the folding process (coils, helices, etc. secondary and tertiary structures) or the types of ligands that bind to proteins may be crucial components for drug design. Exemplary MD simulations that may be used with the invention are described in Alder et al., Studies in Molecular Dynamics. I. General Method. J. Chem. Phys. 31 (2): 459 (1959), Piana et al., Assessing the accuracy of physical models used in protein-folding simulations: quantitative evidence from long molecular dynamics simulations. Current Opinion in Structural Biology (February 2014), and Streett et al., Multiple time-step methods in molecular dynamics. Mol Phys. 35 (3): 639-648 (1978).

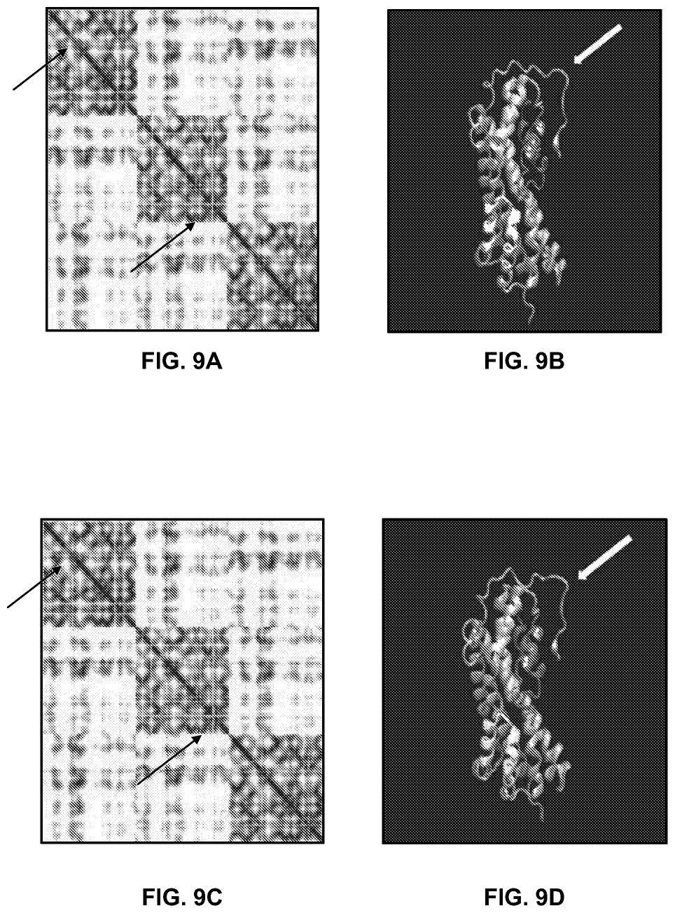

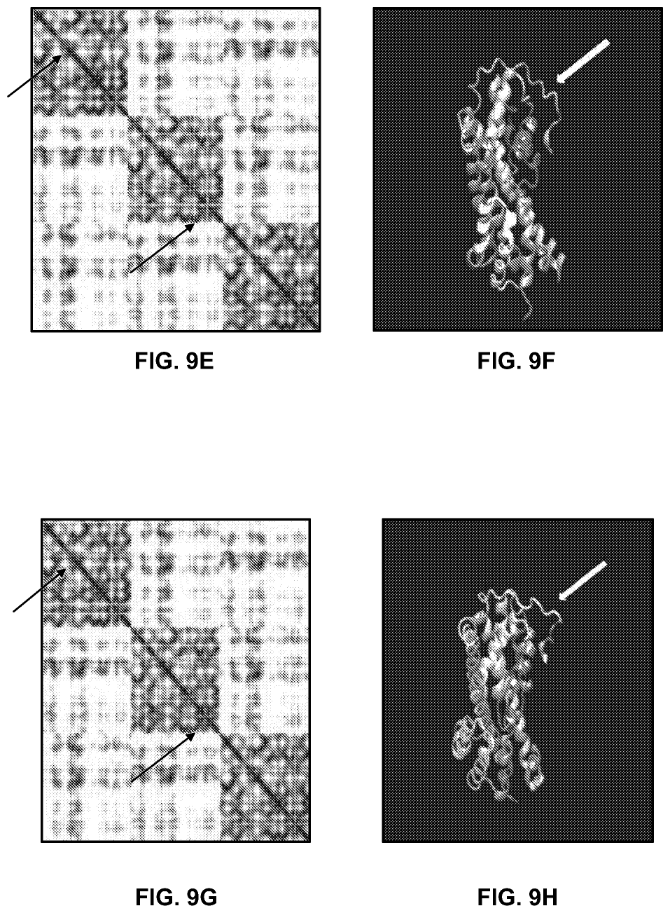

Embodiments of the invention may be used to classify these aspects (i.e., movement, folding etc.) within a molecule and also across molecules similar to an object recognition process performed over many images (e.g., recognizing a cat across many images of animals is similar to recognizing a particular molecular structure across proteins) over a certain time period. Thus, similar to proteins trajectories (simulations of molecule movement over time), certain embodiments of the invention may be configured to compile two or more images similar to video analysis in order to identify the movement of a structure by observing changes in color variations and intensity in the encoded representations as illustrated in FIG. 9A, FIG. 9C, FIG. 9E, and FIG. 9G. For comparison, ribbon diagrams of protein trajectories of the same protein at the same time point are shown in FIG. 9B, FIG. 9D, FIG. 9F, and FIG. 9H. Closed arrows in the encoded representations show changes in the encoded protein images that identify movement of the protein over time. Open arrows in the protein trajectories show corresponding movement of the protein over time.

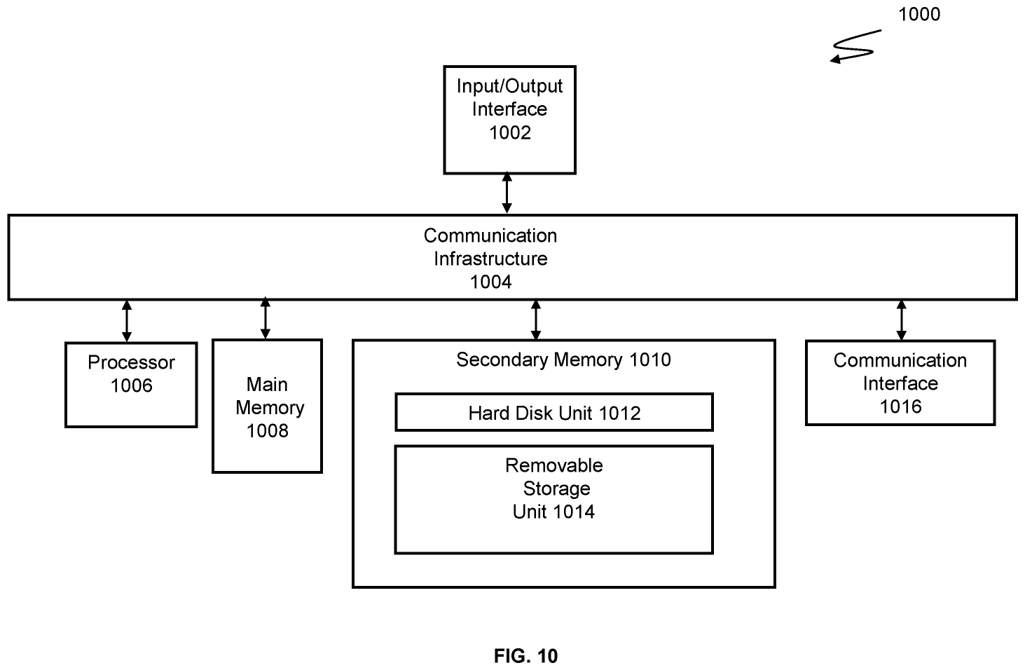

FIG. 10 illustrates an exemplary computer system 1000 that may be used to implement the methods according to the invention. One or more computer systems 800 may carry out the methods presented herein as computer code.

Computer system 1000 includes an input/output display interface 1002 connected to communication infrastructure 1004—such as a bus—which forwards data such as graphics, text, and information, from the communication infrastructure 1004 or from a frame buffer (not shown) to other components of the computer system 1000. The input/output display interface 1002 may be, for example, a keyboard, touch screen, joystick, trackball, mouse, monitor, speaker, printer, any other computer peripheral device, or any combination thereof, capable of entering and/or viewing data.

Computer system 1000 includes one or more processors 1006, which may be a special purpose or a general-purpose digital signal processor that processes certain information. Computer system 1000 also includes a main memory 1008, for example random access memory (“RAM”), read-only memory (“ROM”), mass storage device, or any combination of tangible, non-transitory memory. Computer system 1000 may also include a secondary memory 1010 such as a hard disk unit 1012, a removable storage unit 1014, or any combination of tangible, non-transitory memory. Computer system 1000 may also include a communication interface 1016, for example, a modem, a network interface (such as an Ethernet card or Ethernet cable), a communication port, a PCMCIA slot and card, wired or wireless systems (such as Wi-Fi, Bluetooth, Infrared), local area networks, wide area networks, intranets, etc.

It is contemplated that the main memory 1008, secondary memory 1010, communication interface 1016, or a combination thereof, function as a computer usable storage medium, otherwise referred to as a computer readable storage medium, to store and/or access computer software including computer instructions. For example, computer programs or other instructions may be loaded into the computer system 1000 such as through a removable storage device, for example, a floppy disk, ZIP disks, magnetic tape, portable flash drive, optical disk such as a CD or DVD or Blu-ray, Micro-Electro-Mechanical Systems (“MEMS”), nanotechnological apparatus. Specifically, computer software including computer instructions may be transferred from the removable storage unit 1014 or hard disc unit 1012 to the secondary memory 1010 or through the communication infrastructure 1004 to the main memory 1008 of the computer system 1000.

Communication interface 1016 allows software, instructions and data to be transferred between the computer system 1000 and external devices or external networks. Software, instructions, and/or data transferred by the communication interface 1016 are typically in the form of signals that may be electronic, electromagnetic, optical, or other signals capable of being sent and received by the communication interface 1016. Signals may be sent and received using wire or cable, fiber optics, a phone line, a cellular phone link, a Radio Frequency (“RF”) link, wireless link, or other communication channels.

Computer programs, when executed, enable the computer system 1000, particularly the processor 1006, to implement the methods of the invention according to computer software including instructions.

The computer system 1000 described herein may perform any one of, or any combination of, the steps of any of the methods presented herein. It is also contemplated that the methods according to the invention may be performed automatically or may be invoked by some form of manual intervention.

The computer system 1000 of FIG. 10 is provided only for purposes of illustration, such that the invention is not limited to this specific embodiment. It is appreciated that a person skilled in the relevant art knows how to program and implement the invention using any computer system.

The computer system 1000 may be a handheld device and include any small-sized computer device including, for example, a personal digital assistant (“FDA”), smart hand-held computing device, cellular telephone, or a laptop or netbook computer, hand held console or MP3 player, tablet, or similar hand held computer device, such as an iPad®, iPad Touch® or iPhone®.

While the disclosure is susceptible to various modifications and alternative forms, specific exemplary embodiments of the invention have been shown by way of example in the drawings and have been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

1. A method for prediction a function of a protein comprising:
extracting protein secondary structure information from a primary amino acid sequence using a Ramachadran plot;
expressing a protein tertiary structure information using a distance matrix;
encoding said protein secondary structure information and said protein tertiary structural information into one or more codified color channels to form a representation of said protein secondary structure information and said protein tertiary structural information;
formatting said representation into a fixed-sized encoded representation of said protein secondary structure information and said protein tertiary structural information; and
analyzing said fixed-sized encoded representation to predict protein function.

2. The method of claim 1, wherein said extracting step further includes assigning each amino acid in said primary amino acid sequence a secondary structure selected from the group consisting of an α-helix, a β-strand, a Polyproline PII-helix, a γ′-turn, a γ-turn, a cis-peptide bonds, and indeterminate, based on a constraint of a torsion angle of the amino acid in said primary amino acid sequence.

3. The method of claim 1, wherein said distance matrix for the protein with M Alpha Carbon atoms Cα is a squared matrix D of size M×M, wherein an element D(i, j) corresponds to a distance between Alpha Carbon atoms Cαᵢ and Cαⱼ.

4. The method of claim 1, wherein said representation is an image or tensor.

5. The method of claim 4, wherein said tensor includes a dimension defined by M×M×Z, where M is a number of amino acid residues in the protein and Z is a number of color channels used to encode said protein secondary structure information and said protein tertiary structural information.

6. The method of claim 2, wherein each of said secondary structure is assigned a color.

7. The method of claim 2, wherein said color channels include a red color channel, a green color channel, and a blue color channel, wherein each of said color channels includes a saturation level such that the saturation level of a color is expressed as [1, sd(i, j), sd(i, j)] Åj ∈ D, [sd(i, j), sd(i, j),1], [1, sd(i, j),1], [1,1, sd(i, p], [sd(i, j), 1, 1], [sd(i, j),1, sd(i, j], and [0,0,0], for red, blue, magenta, yellow, cyan, green, and black, respectively.

8. The method of claim 1, wherein said formatting step further includes resizing said representations to produce said fixed-sized encoded representation that is dimensionally consistent.

9. The method of claim 8, wherein said resizing said representations includes the use of a bicubic interpolation.

10. The method of claim 8, wherein said fixed-sized encoded representation is defined as N×N×Z tensor, where N represents a new size of an image, and Z is a number of channels used to encoding said protein secondary structure information and said protein tertiary structural information into a tensor.

11. The method of claim 10, wherein said encoded representation further includes one or more additional protein information selected from the group consisting of electromagnetic charge, residual energy, and hydrophobicity, wherein each of said additional protein information is represented by a separate color channel.

12. The method of claim 1, wherein said analyzing step further includes the use of a convolutional neural network.

13. The method of claim 10, wherein said convolutional neural network analyzes separately each of said codified color channels of said fixed-sized encoded representation using a convolution 2D filter prior to combining each of said color channels into a single representation.

14. A method of determining local and global arrangements of a protein structure during a protein folding simulation or a protein-ligand docking simulation according to claim 10.

15. A computer system for predicting a function of a protein comprising:
one or more processors and a memory storing at least one program for execution by said at least one processor, the at least one program comprising instructions for:
extracting protein secondary structure information from a primary amino acid sequence using a Ramachadran plot;
expressing a protein tertiary structure information using a distance matrix;
encoding said protein secondary structure information and said protein tertiary structural information into one or more codified color channels to form a representation of said protein secondary structure information and said protein tertiary structural information;
formatting said representation into a fixed-sized encoded representation of said protein secondary structure information and said protein tertiary structural information; and
analyzing said fixed-sized encoded representation using a convolutional neural network to predict protein function.
